(19) United States
(12) Patent Application Publication
(10) Pub. No.: US 2016/0326571 A1
(43) Pub. Date: Nov. 10, 2016

(54) METHODS FOR NUCLEIC ACID AMPLIFICATION

(71) Applicant: ATHEROTECH, INC., Birmingham, AL (US)

(72) Inventor: Chen-Hsiung YEH, Birmingham, AL (US)

(73) Assignee: Atherotech, Inc., Birmingham, AL (US)

(21) Appl. No.: 15/033,864

(22) PCT Filed: Oct. 31, 2014
(86) PCT No.: PCT/US2014/063534

§ 371 Date: May 2, 2016

Related U.S. Application Data
(60) Provisional application No. 61/898,192, filed on Oct. 31, 2013.

(51) Int. Cl. C12Q 1/68

(52) U.S. Cl. CPC C12Q 1/6806; C12Q 1/6888; C12Q 2600/156

(57) ABSTRACT

The present disclosure provides novel methods for direct sample nucleic acid amplification with optional detection. The methods of the present disclosure provide for the foregoing without the requirement of nucleic acid purification from the sample. The methods generally comprise diluting a sample containing a nucleic acid target sequence to be amplified to produce a diluted sample, optionally subjecting the diluted sample to processing, either before or after dilution, and performing an amplification reaction on the sample to amplify the nucleic acid target sequence.

### FIELD OF THE DISCLOSURE

The present disclosure relates to methods for nucleic acid amplification and/or detection. Specifically, the present disclosure relates to methods for direct sample nucleic acid amplification and/or detection without the need to purify nucleic acid from the sample.

### BACKGROUND

The use of single nucleotide polymorphism (SNP) genotyping methods is expected to improve our understanding of the genetic basis of complex diseases, personalize diagnosis and risk assessment, help to stratify patients by drug response, and fulfill the potential of pharmacogenomics (Kim, S et al., Annual Review of Biomedical Engineering 2007, 9: 289-320; Klein C, et al.. JAMA 2012, 308(18): 1867-1868). To meet the demands of genomic medicine in the post-genome era, a simplified, high-throughput and cost-effective genotyping assay is required to identify SNPs.

While a number of methods are currently available for identifying SNPs from nucleic acid, polymerase chain reaction (PCR) based methods offer the benefit of high throughput coupled with high selectivity and ease of use. SNP genotyping for PCR based methods can generally be divided into two steps: (i) sample preparation, typically purification of nucleic acid (such as genomic DNA) from a biological specimen (such as blood); and (ii) target allele discrimination and detection. Nucleic acid extraction is a rate-limiting and time-consuming step in the PCR-based genotyping assay conducted in a clinical laboratory, increasing the overall cost and turnaround time of those clinical tests. Furthermore, the various purification procedures are not always efficient and can lead to loss of the target nucleic acid. In addition, the multiple sample manipulations involved increase the risk of cross-contamination.

Peripheral blood is an easily accessible and noninvasive source from which surrogate biomarker genotyping for a variety of diseases is being extensively explored. However, accurate and reproducible SNP genotyping directly from whole blood is made difficult by intrinsic PCR inhibitors, such as heme, hemoglobin, lactoferrin and immunoglobulin G (Al-Soud W A, et al.; J Clin Microbiol 2001, 39:485-493; Al-Soud W A, et al.; , J Clin Microbiol 2000, 38:345-350; Akane, A., et al.; J. Forensic Sci. 1994, 39, 362-372), as well as by inaccessibility of DNA target and impaired fluorescent signal detection by factors in blood (Zhang Z, et al.; J. Mol Diagn. 2010, 12(2): 152-161). In fact, when using the current technologies commonly available for genotyping from blood, the results can be strongly dependent on the selection of DNA isolation techniques themselves (Kramvis A, et al., J Clin Microbiol. 1996;34:2731-2733).

Various methods have been developed to overcome PCR inhibition from blood samples. PCR additives that can relieve the inhibition and enhance amplification have been reported (Bu Y, et al., Anal Biochem 2008, 375:370-372; Nishimura N, et al., Ann Clin Biochem 2000, 37 (Pt 5):674-68014). However, such additives and modified procedures usually address only one aspect of PCR inhibition (such as stabilizing Taq polymerase, increasing efficiency for GC-rich targets or attenuation of inhibition). A blood-resistant mutant of Taq DNA polymerase has also been developed to cope with major PCR inhibitors from blood (Kermekchiev M B, et al., Nucleic Acids Res. 2009;37:e40).

Although whole blood presents unique challenges not found in other systems, the identification of a method for genotyping directly from a sample, such as a blood sample, would address many unmet needs in the field. Such a method has been clinically unavailable to date. The present disclosure addresses the shortcomings of the prior art by providing methods for the direct genotyping of target alleles from a sample (such as blood). The disclosed methods are quick, efficient, and compatible with commonly used blood collection methods. Furthermore, the disclosed methods do not require blood processing, nucleic acid purification, or enzymatic manipulation of the sample.

### SUMMARY OF THE DISCLOSURE

The present disclosure provides methods for amplifying a nucleic acid target sequence from a sample. The disclosed methods are applicable for directly amplifying a target nucleic acid sequence from a sample without the requirement of purifying the target nucleic acid from the sample. In its most broad embodiment, the method comprises diluting a sample, optionally processing the sample to aid in releasing nucleic acid in the sample (where the processing step may occur before dilution, after dilution, or both), and performing an amplification reaction on the sample to amplify the nucleic acid target sequence. In one general embodiment, the method comprises diluting a sample and performing an amplification reaction on the sample to amplify the nucleic acid target sequence. In another general embodiment, the method comprises diluting a sample, processing the sample to aid in releasing nucleic acid from sample, and performing an amplification reaction on the sample to amplify the nucleic acid target sequence. In another general embodiment, the method comprises processing the sample to aid in releasing nucleic acid in the sample, diluting a sample, and performing an amplification reaction on the sample to amplify the nucleic acid target sequence. In the general embodiments above, the method may further comprise analyzing and/or detecting the nucleic acid target sequence.

In a first aspect, the method comprises diluting a sample containing a nucleic acid target sequence to be amplified to produce a diluted sample and performing an amplification reaction on the diluted sample to amplify the nucleic acid target sequence.

In a second aspect, the method comprises diluting a sample containing a nucleic acid target sequence to be amplified to produce a diluted sample, processing the diluted sample to produce a processed sample, and performing an amplification reaction on processed the sample to amplify the nucleic acid target sequence.

In a third aspect, the method comprises processing a sample containing a nucleic acid target sequence to be amplified to produce a processed sample, diluting the processed sample to produce a diluted sample and performing an amplification reaction on the diluted sample to amplify the nucleic acid target sequence.

In a fourth aspect, the method comprises diluting a sample containing a nucleic acid target sequence to be amplified to produce a diluted sample, performing an amplification reaction on the diluted sample to amplify the nucleic acid target sequence to produce an amplified target nucleic acid sequence, and analyzing the amplified target nucleic acid target sequence.

In a fifth aspect, the method comprises diluting a sample containing a nucleic acid target sequence to be amplified to produce a diluted sample, processing the diluted sample to produce a processed sample, performing an amplification reaction on the sample to amplify the nucleic acid target sequence to produce an amplified target nucleic acid sequence, and analyzing the amplified target nucleic acid target sequence.

In a sixth aspect, the method comprises processing a sample containing a nucleic acid target sequence to be amplified to produce a processed sample, diluting the processed sample to produce a diluted sample, performing an amplification reaction on the diluted sample to amplify the nucleic acid target sequence to produce an amplified target nucleic acid, and analyzing the amplified target nucleic acid target sequence.

In a seventh aspect, the method comprises diluting a sample containing a nucleic acid target sequence to be amplified to produce a diluted sample, and performing a PCR -based amplification reaction on the sample to amplify the nucleic acid target sequence to produce an amplified target nucleic acid.

In a eighth aspect, the method comprises diluting a sample containing a nucleic acid target sequence to be amplified to produce a diluted sample, subjecting the diluted sample to at least one freeze-thaw cycle to produce a processed sample, and performing a PCR-based amplification reaction on the processed sample to amplify the nucleic acid target sequence to produce an amplified target nucleic acid.

In a ninth aspect, the method comprises subjecting a sample containing a nucleic acid 5. target sequence to be amplified to at least one freeze-thaw cycle to produce a processed sample, diluting the processed sample a to produce a diluted sample, and performing a PCR-based amplification reaction on the diluted sample to amplify the nucleic acid target sequence to produce an amplified target nucleic acid.

In a tenth aspect, the method comprises diluting a sample containing a nucleic acid target sequence to be amplified to produce a diluted sample, performing a PCR-based amplification reaction on the diluted sample to amplify the nucleic acid target sequence to produce an amplified target nucleic acid, and detecting the amplified target nucleic acid sequence using a labeled nucleic acid construct.

In an eleventh aspect, the method comprises diluting a sample containing a nucleic acid target sequence to be amplified to produce a diluted sample, subjecting the diluted sample to at least one freeze-thaw cycle to produce a processed sample, performing a PCR-based amplification reaction on the processed sample to amplify the nucleic acid target sequence to produce an amplified target nucleic acid, and detecting the amplified target nucleic acid sequence using a labeled nucleic acid construct.

In a twelfth aspect, the method comprises subjecting a sample containing a nucleic acid target sequence to be amplified to at least one freeze-thaw cycle to produce a processed sample, diluting the processed sample to produce a diluted sample, performing a PCR-based amplification reaction on the diluted sample to amplify the nucleic acid target sequence to produce an amplified target nucleic acid, and detecting the amplified target nucleic acid using a labeled nucleic acid construct.

In a thirteenth aspect, the method comprises diluting a sample containing a nucleic acid target sequence to be amplified with water or an alkaline buffer to produce a diluted sample, and performing a real-time PCR-based amplification reaction on the sample to amplify the nucleic acid target sequence to produce an amplified target nucleic acid.

In a fourteenth aspect, the method comprises diluting a sample containing a nucleic acid target sequence to be amplified with water or an alkaline buffer to produce a diluted sample, subjecting the diluted sample to at least one freeze-thaw cycle to produce a processed sample, and performing a real-time PCR-based amplification reaction on the processed sample to amplify the nucleic acid target sequence to produce an amplified target nucleic acid.

In a fifteenth aspect, the method comprises subjecting a sample containing a nucleic acid target sequence to be amplified to at least one freeze-thaw cycle to produce a processed sample, diluting the processed sample with water or an alkaline buffer to produce a diluted sample, and performing a real-time PCR-based amplification reaction on the diluted sample to amplify the nucleic acid target sequence to produce an amplified target nucleic acid.

In a sixteenth aspect, the method comprises diluting a sample containing a nucleic acid target sequence to be amplified with water or an alkaline buffer to produce a diluted sample, performing a real-time PCR-based amplification reaction on the diluted sample to amplify the nucleic acid target sequence to produce an amplified target nucleic acid, and detecting the amplified target nucleic acid using a fluorescently labeled nucleic acid construct.

In a seventeenth aspect, the method comprises diluting a sample containing a nucleic acid target sequence to be amplified with water or an alkaline buffer to produce a diluted sample, subjecting the diluted sample to at least one freeze-thaw cycle to produce a processed sample, performing a real-time PCR-based amplification reaction on the sample to amplify the nucleic acid target sequence to produce an amplified target nucleic acid, and detecting the amplified target nucleic acid using a fluorescently labeled nucleic acid construct.

In an eighteenth aspect, the method comprises subjecting a sample containing a nucleic acid target sequence to be amplified to at least one freeze-thaw cycle to produce a processed sample, diluting the processed sample with water or an alkaline buffer to produce a diluted sample, performing a real-time PCR-based amplification reaction on the diluted sample to amplify the nucleic acid target sequence to produce an amplified target nucleic acid, and detecting the amplified target nucleic acid using a fluorescently labeled nucleic acid construct.

The above presents a simplified summary in order to provide a basic understanding of some aspects of the claimed subject matter. This summary is not an extensive overview. It is not intended to identify key or critical elements or to delineate the scope of the claimed subject matter. Its sole purpose is to present some concepts in a simplified form as a prelude to the more detailed description that is presented later.

### DETAILED DESCRIPTION

The present disclosure provides methods for amplifying a nucleic acid target sequence from a sample. The disclosed methods are applicable for directly amplifying a target nucleic acid from a sample without the requirement of purifying the target nucleic acid from the sample. Therefore, some of the methods of the present disclosure have the advantage over the prior art in that samples can be analyzed directly without the need for purification of the nucleic acid. As a result, the methods of the present disclosure may be performed more efficiently and economically while reducing cross-contamination when processing a large number of samples as compared to the prior art.

In one embodiment, the method comprises diluting a sample to produce a diluted sample and performing an amplification reaction on the diluted sample to amplify the nucleic acid target sequence to produce an amplified target nucleic acid sequence. In another embodiment, the method comprises diluting a sample to produce a diluted sample, processing the diluted sample to aid in releasing nucleic acid from the sample (where the processing step may occur either before or after the dilution step) and performing an amplification reaction on the diluted and/or processed sample to amplify the nucleic acid target sequence to produce an amplified target nucleic acid sequence. In yet another embodiment, the method comprises diluting a sample to produce a diluted sample, processing the diluted sample to aid in releasing nucleic acid in the sample to produce a processed sample and performing an amplification reaction on the processed sample to amplify the nucleic acid target sequence to produce an amplified target nucleic acid sequence. In still another embodiment, the method comprises processing the sample to aid in releasing nucleic acid from the sample to produce a processed sample, diluting the processed sample to produce a diluted sample, and performing an amplification reaction on the diluted sample to amplify the nucleic acid target sequence to produce an amplified target nucleic acid sequence. In any of the foregoing embodiments above, the method may further comprise analyzing the nucleic acid target sequence. In one embodiment, the analyzing step comprises detecting the nucleic acid target sequence. In one embodiment, the method comprises diluting a sample containing a nucleic acid target sequence to be amplified to produce a diluted sample, and performing an amplification reaction on the diluted sample to amplify the nucleic acid target sequence to produce an amplified target nucleic acid sequence.

The methods of the present disclosure may be used with a variety of samples. In one embodiment, the sample is a biological sample. The biological sample may be obtained from any organism (living or dead), including a plant or animal. In one embodiment, the organism is a mammal, including but not limited to a human. A suitable sample may be any that contains a nucleic acid target sequence to be amplified. The sample may contain a cell having the nucleic acid target sequence to be amplified. The sample may contain other types of particles that contain the nucleic acid target sequence, such as a virus, a colloidal particle, an oil droplet, or a micelle. Suitable samples include, but are not limited to, tissue samples (including, but not limited to, a biopsy), blood samples, plasma samples, serum samples, urine samples, saliva samples, buccal swab samples, cell samples and the like. The samples may be pre-processed by methods known in the art prior to use if desired. In one embodiment, the nucleic acid in the sample is not subject to purification procedures and the nucleic acid target sequence is amplified without the need for a purified source of nucleic acid.

Dilution may be performed with a diluent. The diluent used in the methods of the present disclosure may be varied. In one embodiment, the diluent serves to aid in denaturing the nucleic acid prior to the amplification reaction. The diluent is selected so as to not interfere with the amplification reaction or damage the nucleic acid substrate that is subject to amplification. In one embodiment, the diluent is water. In another embodiment, the diluent is a buffer. In another embodiment, the diluent is an amplification acceptable buffer. In another embodiment, the diluent is a PCR acceptable buffer. By amplification acceptable and

PCR acceptable buffer, it is meant that the buffer selected does not interfere with the amplification reaction or damage the nucleic acid substrate. A number of amplification and PCR acceptable buffers are known in the art. Representative amplification acceptable and PCR acceptable buffer include, but are not limited to, Tris buffer, HEPES buffer, phosphate buffer. For example, a commercial buffer used in the particular amplification method may be used in the dilution step.

The pH of the diluent may be any pH desired provided that the pH is compatible with the amplification reaction (for example, the pH is selected to be in a range compatible with the enzymes used in the amplification reaction and not to degrade the nucleic acid substrate). In one embodiment, the diluent is a neutral or alkaline diluent. In one embodiment, the pH is between 3 and 11. In another embodiment, the pH is between 5 and 10. In another embodiment, the pH is between 6 and 9. In another embodiment, the pH is between 8-10. In another embodiment, the pH is 9. In another embodiment, the pH is 7.

In a specific embodiment, the buffer is Tris-EDTA. The pH of the Tris-EDTA buffer may be selected from the ranges specified above. In one embodiment, the pH is between 6 and 9, between 8-10 or 9.

In another specific embodiment, the buffer is HEPES. The pH of the HEPES buffer may be selected from the ranges specified above. In one embodiment, the pH is between 6 and 9, between 8-10 or 9.

In another specific embodiment, the buffer is phosphate buffer. The pH of the phosphate buffer may be selected from the ranges specified above. In one embodiment, the pH is between 6 and 9, between 8-10 or 9.

In another specific embodiment, the diluent is water.

In one embodiment, inhibitors of nucleases and proteases may be added to the diluent to prevent degradation of the target nucleic acid. Any such protease and nuclease inhibitors known in the art may be used.

The sample may be diluted as desired in the methods disclosed. In one embodiment, sample is diluted with diluent in a range of from 1:1 to 1:1,000. In another embodiment, the range of dilution is from 1:5 to 1:500. In another embodiment, the range of dilution is from 1:25 to 1:250. In another embodiment, the range of dilution is from 1:50 to 1:125. In another embodiment, the range of dilution is from 1:75 to 1:100. In a particular embodiment, the dilution is 1:75 or 1:100. In another particular embodiment, the dilution is 1:100. In a further particular embodiment, the range of dilution is 1:75. All of the foregoing dilutions are volume to volume based on the volume of the sample.

In one embodiment, the sample is diluted with diluent at 1:100 or 1:75 and the buffer is Tris-EDTA with a pH of between 8-10.

A suitable amount of the diluted sample, either with or without processing as described herein, is added to the nucleic amplification reaction. By suitable amount of sample, it is meant an amount of sample that produces an amount of amplified nucleic acid target sequence sufficient for the assay being run. The amount of sample added to the nucleic acid amplification reaction may depend on the range of dilution of the sample, the source of the sample, the nucleic acid amplification reaction used, the assay being performed, other factors known in the art and combinations of the foregoing. In one embodiment, the amount of sample added is less than 1%, less than 2%, less than 3%, less than 4%, less than 5%, less than 10% or less than 20% of the total volume of the nucleic acid amplification reaction. In one embodiment, the amount of sample added is less than 5% or less than 10% of the total volume of the nucleic acid amplification reaction. In a particular embodiment, the amount of sample added is from 4% to 8% of the total volume of the nucleic acid amplification reaction. In still another particular embodiment, the amount of sample added is 4% or 7.7% of the total volume of the nucleic acid amplification reaction.

The methods of the present disclosure may be used to amplify any nucleic acid in the sample. The nucleic acid may also be modified, either through normal physiological processes or artificial processes. In one embodiment, the nucleic acid is DNA or RNA. In one embodiment, the DNA includes, but is not limited to, cDNA. In one embodiment, the RNA includes, but is not limited to, mRNA, ribosomal RNA, transfer RNA, small nuclear RNA, micro RNA or small interfering RNA. In one embodiment, the nucleic acid is DNA. In another embodiment, the nucleic acid is methylated, hemimethylated or hydroxymethylated

In the methods disclosed herein, the target nucleic acid may be contained in a coding or non-coding sequence. The nucleic acid target sequence that is amplified may be contained in a larger nucleic acid sequence as is known in the art. For example, the target sequence may be flanked on its 5′, 3′ or both 5′ and 3′ sides by additional nucleic acid sequence that is also amplified in the amplification reaction.

The nucleic acid amplification reaction may be any nucleic acid amplification reaction known in the art. The present disclosure has been shown to work with a variety of amplification reactions. In one embodiment, the amplification reaction is a PCR-based amplification reaction. Such amplification reactions generally involve at least two primers to initiate amplification of the nucleic acid and a heat stable polymerase enzyme to amplify the nucleic acid as well as other components for maximizing efficiency of the reaction and/or that are specific for a given method. Various methods for detecting the amplified nucleic acid may be used if desired. Methods known in the art or as suggested by the manufacturer may be used in carrying out the amplification reaction. In one embodiment, the amplification reaction is a PCR-based amplification method. In another embodiment, the amplification reaction is a real-time PCR-based amplification method. In another embodiment, the amplification reaction is an isothermal multiple displacement amplification (MDA)-based amplification method and may employ the phi29 DNA polymerase. In another embodiment, the amplification reaction is an isothermal rolling circle amplification (RCA) based-amplification method. While a variety of amplification methods may be used, the methods disclosed herein have been shown to provide increased efficiency and/or quantitation that are required in order to process large sample volumes accurately. Representative PCR amplification reactions are known in the art and include, but are not limited to the TaqMan PCR and castPCR platforms (Life Technologies) and the eSensor PCR platform (GenMark), and the ARMS/Scorpion PCR platform (Qiagen/DxS). Therefore, the present disclosure also provides for methods in which the nucleic acid amplification step is a PCR-based or real-time PCR-based amplification reaction.

As discussed herein, the sample may be subject to processing prior to dilution, after dilution, or both before and after the dilution step. In one embodiment, the processing step occurs after the dilution step. In another embodiment, the processing step occurs before the dilution step. The processing step at least aids in the release of nucleic acid from the sample.

Therefore, in another embodiment the method comprises diluting a sample containing a nucleic acid target sequence to be amplified to produce a diluted sample, processing the diluted sample to produce a processed sample, and performing an amplification reaction on the processed sample to amplify the nucleic acid target sequence to produce an amplified nucleic acid target sequence. In still another embodiment, the method comprises processing a sample containing a nucleic acid target sequence to be amplified to produce a processed sample, diluting the processed sample to produce a diluted sample, and performing an amplification reaction on the diluted sample to amplify the nucleic acid target sequence to produce an amplified nucleic acid target sequence.

When a processing step is recited, regardless of whether the processing is performed either before or after dilution, a variety of processing methods may be used. In one embodiment, processing serves to at least lyse cells that may be present in the sample in order to aid in making the nucleic acid accessible for further steps. In one embodiment, the processing steps are compatible with the amplification reaction and does not damage the nucleic acid substrate. Representative processing steps include, but are not limited to, sonication, electroporation, freeze-thaw cycling, vortexing, heating, subjecting the cells to hypo-osmotic conditions, ionic or non-ionic detergents/denaturants, shearing and other membrane disruption techniques. A combination of the foregoing methods may also be used.

In one embodiment, the processing approach used is a freeze-thaw cycle. In such an embodiment, the sample is frozen at a freezing temperature for a period of time and then thawed. Such process disrupts the cell membrane and releases the nucleic acid. The freezing temperature may be any temperature less than 0° C. In one embodiment, the freezing temperature is −20° C., −80° C. or less. In a particular embodiment, the freezing temperature is −80° C. or less. The samples may be maintained at the freezing temperature for a desired period of time, such as for minutes to hours. In one embodiment, the sample is maintained at the freezing temperature for a period of time greater than 10 minutes and less than one hour, such as 10-20 minutes or 20-40 minutes. The frozen samples may be thawed at room temperature or in a water bath or by other means known in the art. In one embodiment, the frozen sample is thawed at room temperature. The cycle may be repeated as necessary.

In one embodiment, inhibitors of nucleases and proteases may be added either before or during processing or before of after dilution to prevent degradation of the target nucleic acid. Any such protease and nuclease inhibitors known in the art may be used.

In one embodiment, the method comprises diluting a sample containing a nucleic acid target sequence to be amplified to produce a diluted sample and performing a PCR-based amplification reaction on the diluted sample to amplify the nucleic acid target sequence to produce an amplified target nucleic acid sequence.

In another embodiment, the method comprises diluting a sample containing a nucleic acid target sequence to be amplified to produce a diluted sample, subjecting the diluted sample to at least one freeze-thaw cycle to produce a processed sample, and performing a PCR-based amplification reaction on the processed sample to amplify the nucleic acid target sequence to produce an amplified target nucleic acid sequence.

In yet another embodiment, the method comprises subjecting a sample containing a nucleic acid target sequence to be amplified to at least one freeze-thaw cycle to produce a processed sample, diluting the processed sample a to produce a diluted sample, and performing a PCR-based amplification reaction on the sample to amplify the nucleic acid target sequence to produce an amplified target nucleic acid sequence.

In a further embodiment, the method comprises diluting a sample containing a nucleic acid target sequence to be amplified with water or an alkaline buffer to produce a diluted sample, and performing a real-time PCR-based amplification reaction on the diluted sample to amplify the nucleic acid target sequence to produce an amplified target nucleic acid sequence.

In another embodiment, the method comprises diluting a sample containing a nucleic acid target sequence to be amplified with water or an alkaline buffer to produce a diluted sample, subjecting the diluted sample to at least one freeze-thaw cycle to produce a processed sample, and performing a real-time PCR-based amplification reaction on the processed sample to amplify the nucleic acid target sequence to produce an amplified target nucleic acid sequence.

In still another embodiment, the method comprises subjecting a sample containing a nucleic acid target sequence to be amplified to at least one freeze-thaw cycle to produce a processed sample, diluting the processed sample with water or an alkaline buffer to produce a diluted sample, and performing a real-time PCR-based amplification reaction on the diluted sample to amplify the nucleic acid target sequence to produce an amplified target nucleic acid sequence.

The amplified target nucleic acid may be subject to analysis or detection after amplification. The amplified target nucleic acid may be analyzed by a variety of means known in the art. Methods of analysis include those associated with various commercial nucleic acid amplification platforms. In one embodiment, the amplified target sequence is analyzed using a nucleic acid construct. The analysis step may involve the use of ancillary reagents as is known in the art. In another embodiment, the amplified target sequence is analyzed using methods for detecting single nucleotide polymorphisms, the methods including, but not limited to, nucleic acid sequencing, gel electrophoresis, capillary array electrophoresis, MALDI-TOF mass spectrometry and other methods. The methods of analysis are not critical to the methods described herein.

The analysis step may include detecting the target nucleic acid. Detection may be performed on the whole amplicon or a subset of the amplicon. In one embodiment, the detecting is carried out using a nucleic acid construct. The nucleic acid construct is a nucleic acid sequence that binds, in one embodiment specifically, to the target nucleic acid sequence. The nucleic acid construct may be any nucleic acid construct known in the art and may be varied depending on the amplification method employed. Suitable nucleic acid constructs include probes, including labeled probes, such as, but not limited to, fluorescently labeled probes, and cassettes; other types of nucleic acid constructs may be used. Representative probes include, but are not limited to, those probes employed in the TaqMan and eSensor PCR methodology. Representative cassettes include, but are not limited to, those cassettes employed in the Invader PCR methodology.

By analysis and detection, it is meant that the target nucleic acid amplified is examined for a desired characteristic. For example, in SNP assays, the analysis step examines the identity of one or more nucleotides at pre-determined positions in the amplified target nucleic acid. Other characteristics may be examined as well, such as the size of the amplified nucleic acid (either with or without cleavage or other manipulation of the amplified target nucleic acid) or the presence or absence of a series of nucleotides within the amplified target nucleic acid.

Any of the methods described herein may be used in conjunction with an analysis and/or detection step.

Therefore, the present disclosure also provides for the above methods in which an analysis and/or detection is included. In one embodiment, the method comprises diluting a sample containing a nucleic acid target sequence to be amplified to produce a diluted sample, performing an amplification reaction on the diluted sample to amplify the nucleic acid target sequence to produce an amplified target nucleic acid target sequence, and analyzing the amplified target nucleic acid target sequence.

In another embodiment, the method comprises diluting a sample containing a nucleic acid target sequence to be amplified to produce a diluted sample, processing the diluted sample to produce a processed sample, and performing an amplification reaction on the processed sample to amplify the nucleic acid target sequence to produce an amplified nucleic acid target sequence, and analyzing the nucleic acid target sequence.

In still another embodiment, the method comprises processing a sample containing a nucleic acid target sequence to be amplified to produce a processed sample, diluting the processed sample to produce a diluted sample, performing an amplification reaction on the diluted sample to amplify the nucleic acid target sequence to produce an amplified nucleic acid target sequence, and analyzing the nucleic acid target sequence.

In still another embodiment, the method comprises diluting a sample containing a nucleic acid target sequence to be amplified to produce a diluted sample, performing a PCR-based amplification reaction on the diluted sample to amplify the nucleic acid target sequence to produce an amplified nucleic acid target sequence, and detecting the nucleic acid target sequence using a labeled nucleic acid construct.

In still another embodiment, the method comprises diluting a sample containing a nucleic acid target sequence to be amplified to produce a diluted sample, subjecting the diluted sample to at least one freeze-thaw cycle to produce a processed sample, performing a PCR-based amplification reaction on the processed sample to amplify the nucleic acid target sequence to produce an amplified nucleic acid target sequence, and detecting the nucleic acid target sequence using a labeled nucleic acid construct.

In still another embodiment, the method comprises subjecting a sample containing a nucleic acid target sequence to be amplified to at least one freeze-thaw cycle to produce a processed sample, diluting the processed sample to produce a diluted sample, performing a PCR-based amplification reaction on the sample to amplify the nucleic acid target sequence to produce an amplified nucleic acid target sequence, and detecting the nucleic acid target sequence using a labeled nucleic acid construct.

In still another embodiment, the method comprises diluting a sample containing a nucleic acid target sequence to be amplified with water or an alkaline buffer to produce a diluted sample, performing a real-time PCR-based amplification reaction on the diluted sample to amplify the nucleic acid target sequence to produce an amplified nucleic acid target sequence, and detecting the nucleic acid target sequence using a fluorescently labeled nucleic acid construct.

In still another embodiment, the method comprises diluting a sample containing a nucleic acid target sequence to be amplified with water or an alkaline buffer to produce a diluted sample, subjecting the diluted sample to at least one freeze-thaw cycle to produce a processed sample, performing a real-time PCR-based amplification reaction on the processed sample to amplify the nucleic acid target sequence to produce an amplified nucleic acid target sequence, and detecting the nucleic acid target sequence using a fluorescently labeled nucleic acid construct.

In still another embodiment, the method comprises subjecting a sample containing a nucleic acid target sequence to be amplified to at least one freeze-thaw cycle to produce a processed sample, diluting the processed sample with water or an alkaline buffer to produce a diluted sample, performing a real-time PCR-based amplification reaction on the diluted sample to amplify the nucleic acid target sequence to produce an amplified nucleic acid target sequence, and detecting the nucleic acid target sequence using a fluorescently labeled nucleic acid construct.

The terms “about” and “approximately” as used in this disclosure shall generally mean an acceptable degree of error or variation for the quantity measured given the nature or precision of the measurements. Typical, exemplary degrees of error or variation are within 20 percent (%), preferably within 10%, and more preferably within 5% of a given value or range of values. For biological systems, the term “about” refers to an acceptable standard deviation of error, preferably not more than 2-fold of a given value. Numerical quantities given herein are approximate unless stated otherwise, meaning that the term “about” or “approximately” can be inferred when not expressly stated.

The term “consisting essentially of” means that, in addition to the recited elements, what is claimed may also contain other elements (steps, structures, ingredients, components, etc.) that do not adversely affect the operability of what is claimed for its intended purpose.

### EXAMPLES

The methods of the present disclosure were incorporated and tested on two commercially available SNP genotyping platforms for various polymorphisms in multiple genes. The TaqMan genotyping platform (Life Technologies, Grand Island, N.Y.) was used to analyze SNPs of the PCSK9 gene. The eSensor genotyping platform (GenMark Dx, Carlsbad, Calif.) was used to analyze SNPs on the Thrombophilia Risk Test (TRT) panel (Factor II, Factor V and MTHFR genes) and the warfarin sensitivity panel (CYP450 2C9 and VKORC1 gene). While the present disclosure demonstrates the novel methods in relation to amplification of target DNA for the detection of various SNPs, the exemplification included herein is not meant to limit the application of the disclosed methods. The methods disclosed may be used to amplify any target nucleic acid without the need for purification of the nucleic acid as a preliminary step. Furthermore, analysis of the amplified target nucleic acid should not be limited to SNP detection as the analysis and detection steps may be varied as would be understood by one of ordinary skill in the art.

### Example 1

Experiments were conducted to determine the optimal dilution factor for whole blood for use in the methods of the present disclosure. Blood samples used in the present disclosure were collected from routine specimens sent to the lab. Blood samples were drawn into an EDTA anticoagulant. Volumes of 2 to 5 ml of blood were generally collected. The blood samples were stored at room temperature (stable for 72 hours) or refrigerated prior to use (stable for 7 days). Frozen, clotted or grossly haemolysed blood samples were discarded.

Whole blood samples were subjected to genotyping for 7 SNP in PCSK9 (Table 2) using PCSK9 genotyping assay kit (Life Technologies, Grand Island, N.Y.). PCR was performed as described below and in Table 1. PCR reactions were performed according to manufacturer's instructions.

Blood samples were obtained and serially diluted (from 1:5 to 1:1000) in water to produce diluted samples. Aliquots of the diluted sample were added directly to the PCR reaction mix and analyzed by real-time PCR. Sample volumes (1 to 5 microliters) tested ranged from 3.2-14.3% (v/v) in the PCR reaction. The results are shown in Table 3.

For samples diluted in water, a bell-shaped dose-response curve was evident, with the 1:100 dilution giving the best amplification as indicated by Ct values (Table 3). Similar results were obtained for samples diluted with alkaline solution (Tris-EDTA, pH 7-9) and for samples subject to a processing step (such as freeze-thaw cycling). Given these results, 100-fold diluted bloods were used in subsequent experiments.

Real-time PCR SNP genotyping of the PCKS9 gene was carried out using the TaqMan allele discrimination method. Briefly, PCR Primers are designed to encompass the PCSK9 SNP(s) rs505151, rs11591147, rs28362286, rs28362263, rs562556, rs7517090, and rs11206510 sites. Two allele-specific TaqMan MGB probes (see Table 2) are designed to detect the two polymorphic alleles of interest (A/G, G/T, A/C, A/G, A/G, A/G, and C/T respectively).

During PCR, each of the MGB probe anneals specifically to its complementary sequence between the forward and reverse primer sites. Detection is achieved with 5′nuclease chemistry by means of exonuclease cleavage and release of a 5′ allele-specific dye label which generates the permanent assay signal. At end of the PCR, the plate is post read by ABI7900HT instrument. Genotype calls for individual sample are made by plotting the normalized intensity of the reporter dyes (VIC/FAM) in each sample on an allelic discrimination plot. An algorithm in the data analysis software assigns individual sample data to a particular cluster and makes the genotype call.

For whole blood, 1-5 μl of the whole blood sample was diluted into 99 μl of the diluent (in the examples below, water or alkaline diluent Tris-EDTA, pH 9.0) and when indicated subjected to freeze-thaw cycling (−80° C. for 10-20 minutes followed by thawing at room temperature). 1-5 μl of the sample was then added to the PCR mix along with 12.5 μl of Taqman Genotyping master mix (2X, Applied Biosystems catalogue number 4324018), 1.25 of SNP genotyping mix (20X, Applied Biosystems catalogue number 3451379), and 10.25 of nuclease-free water (USB Corporation, catalogue number 71786). The PCR conditions are as specified in Table 1.

Plasma and serum samples were processed in an identical manner except that 10 μl of serum or plasma was diluted with 10 μl of diluent (1:1 ratio). The assay reagents and primer/probe sets used in the PCSK9 assays were purchased from Life Technologies (Grand Island, N.Y.) and GenMark Dx (Carlsbad, Calif.), respectively, and performed according to the manufacturer's protocol. The PCSK9 genotyping assay was detected and analyzed using an ABI7900HT real-time PCR instrument (Life Technologies). The PCR conditions used for each test were optimized for direct-blood genotyping and shown in Table 1.

### Example 2

In order to evaluate the accuracy of the disclosed methods, three paired whole blood samples and purified DNA samples were subjected to genotyping for 7 SNPs in PCSK9 gene.

Blood samples were collected as described in Example 1. Purified DNA from whole blood samples was obtained as follows. Extraction of genomic DNA from 0.2 mL of whole EDTA blood was performed using a 96-well Generation Capture Plate kit according to the manufacturer's instruction (Qiagen, Valencia, Calif.). The plate was placed on a TECAN Freedom EVO 150 robotic liquid handling platform (Tecan, San Jose, Calif.) for automatic sample/buffer transfer, binding, washing, and elution. Membrane-bound genomic DNA was eluted in a volume of 200 μl after microwave heating, resulting in a typical yield of 1-2 ug DNA per isolation. DNA samples were then stored at −80° C. until analysis. The corresponding blood samples were stored at 4° C. no more than 7 days before direct genotyping. Paired blood and DNA samples were analyzed side-by-side whenever possible.

PCR was performed as described in Example 1 and Tables 1; PCKS9 SNPs analyzed are those set forth in Table 2. PCR reactions were performed according to manufacturer's instructions.

Blood samples were obtained and serially diluted in (1:100) in water to produce diluted samples. Aliquots of the diluted sample and purified DNA were added directly to the PCR reaction mix and analyzed by real-time PCR. The results are shown in Table 4. As can be see, both whole blood and purified DNA samples showed comparable first-pass call success of 81% (17/21). These results show that the direct-blood PCR efficiency using whole blood as described in the present disclosure are as effective as prior art methods using purified DNA.

To further improve direct-blood PCR efficiency, whole blood was diluted in an alkaline solution (rather than water) and subject to a processing step to enhance cellular nucleic acid release. In this experiment, whole blood samples were diluted 1:100 in Tris-EDTA (pH 7-9) to produce a diluted sample. The diluted samples were subject to a processing step(in this example freeze-thaw cycling) by placing the diluted sample at −80° C. for 10-20 min. and allowing the sample to thaw at room temperature. The processed whole blood sample and purified DNA sample (obtained as described above) were added directly to the PCR reaction mix and analyzed by real-time PCR as described above.

The results are shown in Table 5. Initial genotyping of 2 PCSK9 polymorphisms (rs562556 and rs11591147) on a cohort of 5 matched (unpurified) whole blood and purified

DNA samples showed a perfect 100% concordance (samples 4-8, Table 5). Further experiments using another 5 paired whole blood and purified DNA samples on all 7 PCSK9 SNPs (samples 9-13, Table 5) displayed again high concordance between both groups except 2 SNPs: rs28362286 and rs11591147. The SNP rs28362286 produced a negative result on control purified DNA or whole blood samples, suggesting that for some samples, certain polymorphisms may not be accessible for amplification due to the position effect on the chromosome. Nevertheless, the overall first-pass success rate for direct-blood genotyping in this pool is 86.7% (39/45).

In order to further evaluate the accuracy of the disclosed methods using a different nucleic acid amplification platform, the direct sample amplification method was tested on the eSensor platform using the thrombophilia risk test (TRT) genotyping and Warfarin sensitivity genotyping assay (GenMark Dx). Both the TRT and warfarin sensitivity test are FDA approved IVD assays. The assay reagents, primer/probe sets used in the TRT and warfarin sensitivity genotyping assay were purchased from GenMark Dx (Carlsbad, Calif.) and performed according to the manufacturer's protocol. The TRT and warfarin sensitivity genotyping assays were detected and analyzed by eSensor XT-8 (GenMark Dx). The PCR conditions used for each test were optimized for direct-blood genotyping and shown in Table 1.

The eSensor technology (GenMark Dx, Carlsbad, Calif.) is a real-time PCR based method for determining SNP. A patient sample is obtained and, according to the methods of the prior art, DNA extraction is performed. PCR is performed to amplify patient DNA, referred to as target DNA. An exonuclease reaction is performed to create single stranded DNA. Multiplex detection and result reporting are performed using the XT-8 system. The target DNA is mixed with the signal probe solution. If the applicable target DNA is present, hybridization to the signal probes occurs immediately. The solution is pumped through the XT-8 cartridge's microfluidic chamber and the target DNA/signal probe complex completes the reaction with the pre-assembled capture probe. The target DNA is detected using electrochemical detection.

For TRT genotype testing, 6 matched whole blood and purified DNA samples were used. Whole blood samples were obtained and processed as described in Example 1 (whole blood diluted with alkaline buffer and processed using freeze-thaw cycling with the exception that the dilution factor was 1:75 sample to diluent). The processed whole blood sample (1 to 5 microliters) was added directly to the PCR reaction mix. In addition, 6 matched purified DNA samples (purified by TECAN onto QIAGEN Capture Plates as described in Example, 2) were also analyzed. All samples were analyzed using the eSensor real-time PCR platform. The SNPs analyzed and the reaction conditions used are as shown in Table 1. eSensor PCR was carried out as per manufactures instructions. The results are shown in Table 6. Similar to PCSK9 genotyping, the results on Factor II, Factor V, MTHFR 677 and MTHFR1298 gene polymorphisms indicated a strong concordance (21/24, 87.5%) between whole blood and purified DNA samples.

For warfarin sensitivity genotype testing, 4 matched whole blood and purified DNA samples were used. Whole blood samples were obtained and processed as described in Example 2 (whole blood diluted 1:100 with alkaline buffer and processed using freeze-thaw cycling). The processed whole blood sample was added directly to the PCR reaction mix. In addition, 4 matched purified DNA samples (purified by TECAN onto QIAGEN Capture Plates as described in Example 2) were also analyzed. All samples were analyzed using the eSensor real-time PCR platform. The SNPs analyzed and the reaction conditions used are as shown in Table 1. eSensor PCR was carried out as per manufactures instructions. The results are shown in Table 7. Gene polymorphism analysis of CYP450 2C9*2, *3 and VKORC1 gene alleles using diluted blood revealed a perfect 100% (12/12) concordance to those results from purified DNA.

The results in Tables 6 and 7 show that the methods of the present disclosure may be used on a variety of PCR platforms with good results.

Table 8 provides a summary of the results from the direct genotyping methods disclosed for the PCSK9, TRT and warfarin sensitivity assays. As can be seen, the concordance rates were 84.8% for PCSK9, 87.5% for TRT genotyping and 100% for warfarin sensitivity. These results show that direct sample amplification of nucleic acid using the methods disclosed is comparable to the state of the art methods using purified DNA.

### Example 3

### Intra-Assay Variation

In order to evaluate the intra-assay precision of the disclosed direct sample genotyping methods, five EDTA whole blood samples were obtained and processed as described in Example 2 (whole blood diluted 1:100 with alkaline buffer and processed using freeze-thaw cycling). The processed whole blood sample was added directly to the PCR reaction mix and analyzed by real-time PCR using the PCSK9 polymorphism assay as described in Example 2. Each sample was run in 3 replicates on all 7 SNPs of PCSK9 (as shown in Table 1) to confirm consistency of the method within replicates. The results are shown in Table 9. As can be seen, the concordance rate was 100%. Three purified DNA samples (purified by TECAN onto QIAGEN Capture Plates as described in Example 2) were also analyzed for all 7 SNPs of PCSK9. The results were identical (data not shown).

### Example 4

### Inter-Assay Variation

In order to evaluate the intra-assay precision of the disclosed direct sample genotyping methods, five EDTA whole blood were obtained and processed as described in Example 2 (whole blood diluted 1:100 with alkaline buffer and processed using freeze-thaw cycling). The processed whole blood sample was added directly to the PCR reaction mix and analyzed by real-time PCR using the PCSK9 polymorphism assay as described in Example 2. Each sample was setup and run on 3 different days on all 7 SNPs of PCSK9 (as shown in Table 1). Each sample was run under the same conditions three consecutive days on all 7 SNPs of PCSK9 to confirm consistency of the method over time. The results are shown in Table 10. As can be seen, the concordance rate was 100%. Two purified DNA samples (purified by TECAN onto QIAGEN Capture Plates as described in Example 2) were also analyzed for all 7 SNPs of PCSK9. The results were identical (data not shown).

### Example 5

### Analysis of Clinical Samples

The methods of the present disclosure were further used to analyze a larger number of clinical samples. Accuracy study using “direct-blood” method was performed on the 7 PCSK9 gene polymorphisms side-by-side with purified DNA from 50 patients (total 350 data points).

Whole blood was obtained and processed as described in Example 1 (whole blood diluted 1:100 with alkaline buffer and processed using freeze-thaw cycling). The processed whole blood sample was added directly to the PCR reaction mix and analyzed by real-time PCR using the PCSK9 polymorphism assay as described in Example 2. Matching purified DNA samples (purified by TECAN onto QIAGEN Capture Plates as described in Example 2) were also analyzed as described above. Conclusion: Direct-blood genotyping showed 99.4% sensitivity and 100% specificity as compared to purified DNA in this cohort (Table 11).

### Example 6

### Analysis of Plasma and Serum Samples

In addition to whole blood samples, a total of 50 matched specimens (30 matched plasma and DNA; 20 matched serum and DNA) were used to validate the “direct-sample” genotyping method on 7 SNPs of PCSK9 gene. As shown here, most samples obtained interpretable genotyping and showed high degree of concordance to the corresponding DNA samples (Table 12 & 13). Therefore, direct-plasma and direct-serum are both validated sample types for direct-sample PCSK9 genotyping assay.

Conclusion The above-examples demonstrate the utility of direct sample nucleic acid amplification methods disclosed. Allele genotypes were successfully called at the concentrations of 0.04% blood for PCSK9 SNPs, and at 0.1% blood for TRT and Warfarin sensitivity polymorphisms. The overall concordance of direct genotyping from whole blood compared to purified DNA on 66 PCSK9, 24 TRT and 12 Warfarin genotype calls are 84.8% (56/66), 87.5% (21/24) and 100% (12/12), respectively (Table 7). The direct-sample genotyping method is simple and cost-effective since it does not require isolation of genomic DNA from the patient's sample. In addition to its simplicity and low-cost, this method also reduces the probability of sample carry-over which may occur in the process of DNA extraction. The direct-sample genotyping is an ideal “primary” method for large population genotype screening in a clinical laboratory. The disclosed methods can be applied to a broad range of clinical genetic tests with the advantages of immediate sample testing, improving workflow, and lowering workload, costs and turnaround time.

### REFERENCES

1. Kim, S and Misra A. SNP Genotyping: Technologies and Biomedical Applications. Annual Review of Biomedical Engineering 2007, 9: 289-320
2. Klein C, Lohmann K, Ziegler A. The promise and limitations of genome-wide association studies. JAMA 2012, 308(18): 1867-1868
3. Al-Soud W A, Radstrom P: Purification and characterization of PCR-inhibitory components in blood cells, J Clin Microbiol 2001, 39:485-493
4. Al-Soud W A, Jonsson L J, Radstrom P: Identification and characterization of immunoglobulin G in blood as a major inhibitor of diagnostic PCR, J Clin Microbiol 2000, 38:345-350
5. Akane, A., Matsubara, K., Nakamura, H., Takahashi, S. and Kimura, K. (1994) Identification of the heme compound copurified with deoxyribonucleic acid (DNA) from bloodstains, a major inhibitor of PCR amplification. J. Forensic Sci., 39, 362-372
6. Zhang Z, Kermekchiev M K, Barnes W M. Direct DNA Amplification from Crude Clinical Samples Using a PCR Enhancer Cocktail and Novel Mutants of Taq. J. Mol Diagn. 2010, 12(2): 152-161
7. Kramvis A, Bukofzer S, Kew M C. Comparison of hepatitis B virus DNA extractions from serum by the QIAamp blood kit, GeneReleaser, and the phenol-chloroform method. J Clin Microbiol. 1996;34:2731-2733
8. Bu Y, Huang H, Zhou G: Direct PCR from human whole blood and filter-paper-dried blood by using a PCR buffer with a higher pH, Anal Biochem 2008, 375:370-372
9. Nishimura N, Nakayama T, Tonoike H, Kojima K, Kato S: Direct PCR from whole blood without DNA isolation, Ann Clin Biochem 2000, 37 (Pt 5):674-68014

10. Kermekchiev M B, Kirilova L I, Vail E E, Barnes W M. Mutants of Taq DNA polymerase resistant to PCR inhibitors allows DNA amplification from whole blood and crude soil samples. Nucleic Acids Res. 2009;37:e40.

TABLE 1
General PCR Reaction Conditions

| Gene Name | SNP ID | Ref. SNP Allele | PCR Conditions |
|---|---|---|---|
| PCSK9 | rs562556 | A > G | 95° C.^{10 min} × 1 cycle; |
| | | | (92° C.^{15 sec} − 60° C.^{1 min}) × 50 cycles |
| PCSK9 | rs505151 | A > G | 95° C.^{10 min} × 1 cycle; |
| | | | (92° C.^{15 sec} − 60° C.^{1 min}) × 50 cycles |
| PCSK9 | rs11206510 | T > C | 95° C.^{10 min} × 1 cycle; |
| | | | (92° C.^{15 sec} − 60° C.^{1 min}) × 50 cycles |
| PCSK9 | rs11591147 | G > T | 95° C.^{10 min} × 1 cycle; |
| | | | (92° C.^{15 sec} − 60° C.^{1 min}) × 50 cycles |
| PCSK9 | rs28362286 | C > A | 95° C.^{10 min} × 1 cycle; |
| | | | (92° C.^{15 sec} − 60° C.^{1 min}) × 50 cycles |
| PCSK9 | rs28362263 | G > A | 95° C.^{10 min} × 1 cycle; |
| | | | (92° C.^{15 sec} − 60° C.^{1 min}) × 50 cycles |
| PCSK9 | rs7517090 | G > A | 95° C.^{10 min} × 1 cycle; |
| | | | (92° C.^{15 sec} − 60° C.^{1 min}) × 50 cycles |
| Factor II | 20210 | G > A | 95° C.^{4 min} × 1 cycle; |
| (Prothrombin) | | | (95° C.^{25 sec} − 60° C.^{30 sec} − 72° C.^{25 sec}) × |
| | | | 35 cycles |
| Factor V | 1691 | G > A | 95° C.^{4 min} × 1 cycle; |
| (Leiden) | | | (95° C.^{25 sec} − 60° C.^{30 sec} − 72° C.^{25 sec}) × |
| | | | 35 cycles |
| MTHFR | 677 | C > T | 95° C.^{4 min} × 1 cycle; |
| | 1298 | A > C | (95° C.^{25 sec} − 60° C.^{30 sec} − 72° C.^{25 sec}) × |
| | | | 35 cycles |
| CYP450 | 430 | C > T | 95° C.^{4 min} × 1 cycle; |
| 2C9*2, *3 | 1075 | A > C | (93° C.^{45 sec} − 56° C.^{45 sec} − 68° C.^{45 sec}) × |
| | | | 39 cycles; |
| | | | 68° C.^{7 min} × 1 cycle |
| VKORC1 | −1639 | G > A | 95° C.^{4 min} × 1 cycle; |
| | | | (93° C.^{45 sec} − 56° C.^{45 sec} − 68° C.^{45 sec}) × |
| | | | 39 cycles; |
| | | | 68° C.^{7 min} × 1 cycle |

TABLE 2
Sequences of PCKS9 gene polymorphisms

| Gene Name | SNP ID | Sequence Adjacent to Allele SNP |
|---|---|---|
| PCSK9 | rs562556 | GGGGCCTACACGGATGGCCACAGCC[A/G]TCGCCCGCTGCGC |
| | | CCCAGATGAGGA |
| PCSK9 | rs505151 | AGCACTACAGGCAGCACCAGCGAAG[A/G]GGCCGTGACAGCC |
| | | GTTGCCATCTGC |
| PCSK9 | rs11206510 | AAGGATATAGGGAAAACCTTGAAAG[C/T]GATGTCTGTGGTG |
| | | GCCGTCTTTGGC |
| PCSK9 | rs11591147 | TACGAGGAGCTGGTGCTAGCCTTGC[G/T]TTCCGAGGAGGAC |
| | | GGCCTGGCCGAA |
| PCSK9 | rs28362286 | CCGTGACAGCCGTTGCCATCTGCTG[A/C]CGGAGCCGGCACCT |
| | | GGCGCAGGCCT |
| PCSK9 | rs28362263 | GGTACTGACCCCCAACCTGGTGGCC[A/G]CCCTGCCCCCCAGC |
| | | ACCCATGGGGC |
| PCSK9 | rs7517090 | GAGTGTGGCCTGTGCAGAAGGGACC[A/G]AGGCTGGTGAGAC |
| | | CAGGAGGGCCTG |

TABLE 3
Determination of Optimal Dilution Values

| | Ct Value | | |
|---|---|---|---|
| Dilution Factor | Sample #1 | Sample #2 | Sample #3 |
| 0 | UD | UD | UD |
| 5 | UD | UD | UD |
| 10 | UD | UD | 37.97 |
| 25 | 36.90 | UD | 33.65 |
| 50 | 34.99 | 36.49 | 34.45 |
| 75 | 35.69 | 35.19 | 33.17 |
| 100 | 29.44 | 34.98 | 33.82 |
| 200 | 34.23 | | |
| 400 | 36.59 | | |
| 600 | 35.64 | | |
| 800 | 38.92 | | |
| 1000 | 39.96 | | |

*Undetermined value

TABLE 4
Comparison of results from paired DNA and blood samples
in TaqMan PCSK9 genotyping assay (water as diluent)

| | | | |
|---|---|---|---|
| SNP ID | DNA Control | Purified DNA | Diluted Blood |

Sample 1

| | | | |
|---|---|---|---|
| rs562556 | AA | AA | AA |
| rs28362286 | —* | — | CC |
| rs11591147 | GG | — | GG |
| rs505151 | AA | AA | — |
| rs7517090 | GG | — | GG |
| rs11206510 | CT | TT | TT |
| rs28362263 | GG | GG | GG |

Sample 2

| | | | |
|---|---|---|---|
| rs562556 | AA | AA | AA |
| rs28362286 | — | CC | — |
| rs11591147 | GG | GG | GG |
| rs505151 | AA | AA | — |
| rs7517090 | GG | GG | GG |
| rs11206510 | CT | TT | TT |
| rs28362263 | GG | GG | GG |

Sample 3

| | | | |
|---|---|---|---|
| rs562556 | AA | AA | — |
| rs28362286 | — | CC | CC |
| rs11591147 | GG | GG | GG |
| rs505151 | AA | AA | AA |
| rs7517090 | GG | GG | GG |
| rs11206510 | CT | TT | TT |
| rs28362263 | GG | — | GG |

*No call

TABLE 5
Comparison of results from paired DNA and blood samples in TaqMan
PCSK9 genotyping assay (alkaline diluent + cold shock)

| | | | |
|---|---|---|---|
| SNP ID | DNA Control | Purified DNA | Diluted Blood |

Sample 4

| | | | |
|---|---|---|---|
| rs562556 | AA | AA | AA |
| rs11591147 | GG | GG | GG |

Sample 5

| | | | |
|---|---|---|---|
| rs562556 | AA | AG | AG |
| rs11591147 | GG | GG | GG |

Sample 6

| | | | |
|---|---|---|---|
| rs562556 | AA | AG | AG |
| rs11591147 | GG | GG | GG |

Sample 7

| | | | |
|---|---|---|---|
| rs562556 | AA | AG | AG |
| rs11591147 | GG | GG | GG |

Sample 8

| | | | |
|---|---|---|---|
| rs562556 | AA | AA | AA |
| rs11591147 | GG | GG | GG |

Sample 9

| | | | |
|---|---|---|---|
| rs562556 | AA | AA | AA |
| rs28362286 | —* | CC | CC |
| rs11591147 | GG | GG | — |
| rs505151 | AA | AA | AA |
| rs7517090 | GG | GG | GG |
| rs11206510 | CT | TT | TT |
| rs28362263 | GG | GG | GG |

Sample 10

| | | | |
|---|---|---|---|
| rs562556 | AA | AA | AA |
| rs28362286 | — | AC | AC |
| rs11591147 | GG | GG | — |
| rs505151 | AA | AA | AA |
| rs7517090 | GG | GG | GG |
| rs11206510 | CT | TT | TT |
| rs28362263 | GG | GG | GG |

Sample 11

| | | | |
|---|---|---|---|
| rs562556 | AA | AA | AA |
| rs28362286 | — | AC | — |
| rs11591147 | GG | GG | GG |
| rs505151 | AA | AA | AA |
| rs7517090 | GG | GG | GG |
| rs11206510 | CT | TT | TT |
| rs28362263 | GG | GG | GG |

Sample 12

| | | | |
|---|---|---|---|
| rs562556 | AA | AA | AA |
| rs28362286 | — | CC | — |
| rs11591147 | GG | GG | GG |
| rs505151 | AA | AA | AA |
| rs7517090 | GG | GG | GG |
| rs11206510 | CT | TT | TT |
| rs28362263 | GG | GG | GG |

Sample 13

| | | | |
|---|---|---|---|
| rs562556 | AA | AA | AA |
| rs28362286 | — | AC | — |
| rs11591147 | GG | GG | — |
| rs505151 | AA | AA | AA |
| rs7517090 | GG | GG | GG |
| rs11206510 | CT | CT | CT |
| rs28362263 | GG | GG | GG |

*No call

TABLE 6
Comparison of results from paired DNA and blood samples
in TRT genotyping assay (alkaline diluent + cold shock)

| | | |
|---|---|---|
| SNP ID | Purified DNA | Diluted Blood |

Sample 1

| | | |
|---|---|---|
| Factor II 20210G > A | GG | GG |
| Factor V 1691G > A | GG | GG |
| MTHFR 677C > T | CT | CT |
| MTHFR 1298A > C | AA | AA |

Sample 2

| | | |
|---|---|---|
| Factor II 20210G > A | GG | GG |
| Factor V 1691G > A | GG | GG |
| MTHFR 677C > T | CT | — |
| MTHFR 1298A > C | AA | AA |

Sample 3

| | | |
|---|---|---|
| Factor II 20210G > A | GG | GG |
| Factor V 1691G > A | GG | GG |
| MTHFR 677C > T | CC | CC |
| MTHFR 1298A > C | AA | AA |

Sample 4

| | | |
|---|---|---|
| Factor II 20210G > A | GG | GG |
| Factor V 1691G > A | GG | GG |
| MTHFR 677C > T | CC | CC |
| MTHFR 1298A > C | CC | CC |

Sample 5

| | | |
|---|---|---|
| Factor II 20210G > A | GG | — |
| Factor V 1691G > A | GG | GG |
| MTHFR 677C > T | CC | — |
| MTHFR 1298A > C | AA | AA |

Sample 6

| | | |
|---|---|---|
| Factor II 20210G > A | GG | GG |
| Factor V 1691G > A | GG | GG |
| MTHFR 677C > T | CC | CC |
| MTHFR 1298A > C | AC | AC |

*No call

TABLE 7
Comparison of results from paired DNA and blood samples in
Warfarin genotyping assay (alkaline diluent + cold shock)

| | | |
|---|---|---|
| | Purified | |
| SNP ID | DNA | Diluted Blood |

Sample 1

| | | |
|---|---|---|
| CYP450 2C9 | *1/*2 | *1/*2 |
| VKORC1 | AA | AA |

Sample 2

| | | |
|---|---|---|
| CYP450 2C9 | *1/*1 | *1/*1 |
| VKORC1 | GA | GA |

Sample 3

| | | |
|---|---|---|
| CYP450 2C9 | *1/*2 | *1/*2 |
| VKORC1 | GA | GA |

Sample 4

| | | |
|---|---|---|
| CYP450 2C9 | *1/*2 | *1/*2 |
| VKORC1 | AA | AA |

TABLE 8
Summary of direct-blood genotyping results

| | Success Rate | No. Genotype | |
|---|---|---|---|
| Test Name | (% Concordance) | Analysis | Assay Platform |
| PCSK9 | 84.8 | 66 | TaqMan genotyping |
| TRT | 87.5 | 24 | GenMark eSensor |
| Warfarin | 100.0 | 12 | GenMark eSensor |

TABLE 9

| | | | | |
|---|---|---|---|---|
| | | | | % |
| SNP ID | Replicate 1 | Replicate 2 | Replicate 3 | Concordance |

Sample 1

| | | | | |
|---|---|---|---|---|
| rs11206510 | TT | TT | TT | 100 |
| rs11591147 | GG | GG | GG | 100 |
| rs28362263 | GG | GG | GG | 100 |
| rs28362286 | CC | CC | CC | 100 |
| rs505151 | AA | AA | AA | 100 |
| rs562556 | AA | AA | AA | 100 |
| rs7517090 | GG | GG | GG | 100 |

Sample 2

| | | | | |
|---|---|---|---|---|
| rs11206510 | TT | TT | TT | 100 |
| rs11591147 | GG | GG | GG | 100 |
| rs28362263 | GG | GG | GG | 100 |
| rs28362286 | CC | CC | CC | 100 |
| rs505151 | AA | AA | AA | 100 |
| rs562556 | AA | AA | AA | 100 |
| rs7517090 | GG | GG | GG | 100 |

Sample 3

| | | | | |
|---|---|---|---|---|
| rs11206510 | TT | TT | TT | 100 |
| rs11591147 | GG | GG | GG | 100 |
| rs28362263 | GG | GG | GG | 100 |
| rs28362286 | CC | CC | CC | 100 |
| rs505151 | AA | AA | AA | 100 |
| rs562556 | AA | AA | AA | 100 |
| rs7517090 | GG | GG | GG | 100 |

Sample 4

| | | | | |
|---|---|---|---|---|
| rs11206510 | TT | TT | TT | 100 |
| rs11591147 | GG | GG | GG | 100 |
| rs28362263 | GG | GG | GG | 100 |
| rs28362286 | CC | CC | CC | 100 |
| rs505151 | AA | AA | AA | 100 |
| rs562556 | AA | AA | AA | 100 |
| rs7517090 | GG | GG | GG | 100 |

Sample 5

| | | | | |
|---|---|---|---|---|
| rs11206510 | TT | TT | TT | 100 |
| rs11591147 | GG | GG | GG | 100 |
| rs28362263 | GG | GG | GG | 100 |
| rs28362286 | CC | CC | CC | 100 |
| rs505151 | AA | AA | AA | 100 |
| rs562556 | A/G | A/G | A/G | 100 |
| rs7517090 | GG | GG | GG | 100 |

TABLE 10

| | | | | |
|---|---|---|---|---|
| | | | | % |
| SNP ID | Day 1 | Day 2 | Day 3 | Concordance |

Sample 1

| | | | | |
|---|---|---|---|---|
| rs11206510 | TT | TT | TT | 100 |
| rs11591147 | GG | GG | GG | 100 |
| rs28362263 | GG | GG | GG | 100 |
| rs28362286 | CC | CC | CC | 100 |
| rs505151 | AA | AA | AA | 100 |
| rs562556 | AA | AA | AA | 100 |
| rs7517090 | GG | GG | GG | 100 |

Sample 2

| | | | | |
|---|---|---|---|---|
| rs11206510 | TT | TT | TT | 100 |
| rs11591147 | GG | GG | GG | 100 |
| rs28362263 | GG | GG | GG | 100 |
| rs28362286 | CC | CC | CC | 100 |
| rs505151 | AA | AA | AA | 100 |
| rs562556 | AA | AA | AA | 100 |
| rs7517090 | GG | GG | GG | 100 |

Sample 3

| | | | | |
|---|---|---|---|---|
| rs11206510 | TT | TT | TT | 100 |
| rs11591147 | GG | GG | GG | 100 |
| rs28362263 | GG | GG | GG | 100 |
| rs28362286 | CC | CC | CC | 100 |
| rs505151 | AA | AA | AA | 100 |
| rs562556 | AA | AA | AA | 100 |
| rs7517090 | GG | GG | GG | 100 |

Sample 4

| | | | | |
|---|---|---|---|---|
| rs11206510 | TT | TT | TT | 100 |
| rs11591147 | GG | GG | GG | 100 |
| rs28362263 | GG | GG | GG | 100 |
| rs28362286 | CC | CC | CC | 100 |
| rs505151 | AA | AA | AA | 100 |
| rs562556 | AA | AA | AA | 100 |
| rs7517090 | GG | GG | GG | 100 |

Sample 5

| | | | | |
|---|---|---|---|---|
| rs11206510 | TT | TT | TT | 100 |
| rs11591147 | GG | GG | GG | 100 |
| rs28362263 | GG | GG | GG | 100 |
| rs28362286 | CC | CC | CC | 100 |
| rs505151 | AA | AA | AA | 100 |
| rs562556 | A/G | A/G | A/G | 100 |
| rs7517090 | GG | GG | GG | 100 |

TABLE 11

Direct Blood

| DNA | Homozygous | Heterozygous |
|---|---|---|
| Homozygous | 328 | 0 |
| Heterozygous | 2 | 20 |
| Sensitivity | 99.4% | |
| Specificity | 100% | |

TABLE 12

Direct Plasma*

| DNA | Homozygous | Heterozygous |
|---|---|---|
| Homozygous | 190 | 4 |
| Heterozygous | 3 | 6 |
| Sensitivity | 97.9% | |
| Specificity | 66.7% | |

*Two plasma and 4 DNA samples failed to make calls; one sample was called GG in DNA while was AA in matched plasma.

TABLE 13

Direct Serum

| DNA | Homozygous | Heterozygous |
|---|---|---|
| Homozygous | 121 | 7 |
| Heterozygous | 3 | 9 |
| Sensitivity | 94.5% | |
| Specificity | 75.0% | |

1. A method of amplifying a nucleic acid target sequence from a sample without purifying wthe nucleic acids from the sample, the method comprising:
(a) diluting the sample by at least 1:50; and
(b) performing an amplification reaction on the sample to amplify the nucleic acid target sequence;
wherein nucleic acids are not purified prior to performing either of steps (a) or (b).

2. The method of claim 1, wherein the method is an in vitro method.

3. The method of claim 1, further comprising (c) processing the sample before or after step (a).

4. The method of claim 1, further comprising (c) processing the sample before or after step (a) by lysing or permeabilizing a cell in the sample.

5. The method of claim 1, further comprising (c) processing the sample before or after step (a) by lysing or permeabilizing cells in the sample by a process selected from the group consisting of: freeze-thaw cycling, sonicating, heating, shearing, vortexing, introducing detergents, introducing denaturants, and exposing the cells to hypo-osmotic conditions.

6. The method of claim 1, further comprising (c) processing the sample before or after step (a) by subjecting the sample to at least one freeze-thaw cycle.

7. The method of any one of claim 1, further comprising (c) processing the sample before or after step (a) by subjecting the sample to at least one freeze-thaw cycle by freezing the sample for at least 10 minutes.

8. The method of claim 1, further comprising (c) processing the sample before or after step (a) by subjecting the sample to at least one freeze-thaw cycle by freezing the sample for 10-60 minutes.

9. The method of claim 1, further comprising (c) processing the sample before or after step (a) by subjecting the sample to at least one freeze-thaw cycle by freezing the sample for 10-20 minutes.

10. The method of claim 1, further comprising (c) processing the sample before or after step (a) by subjecting the sample to at least one freeze-thaw cycle at −20 C or lower.

11. The method of claim 1, further comprising (c) processing the sample before or after step (a) by subjecting the sample to at least one freeze-thaw cycle at −80 C or lower.

12. The method of claim 1, further comprising (c) processing the sample before or after step (a) by subjecting the sample to at least one freeze-thaw cycle at −80 C for 10-20 minutes followed by thawing.

13. The method of claim 1, further comprising (c) processing the sample to release nucleic acids after step (a).

14. The method of claim 1, wherein step (a) comprises diluting the sample by at least about 1:75.

15. The method of claim 1, wherein step (a) comprises diluting the sample by at least about 1:100.

16. (canceled)

17. The method of claim 1, wherein step (a) comprises diluting the sample with a diluent selected from the group consisting of: water and an alkaline buffer.

18. The method of claim 1, wherein step (a) comprises diluting the sample with an alkaline buffer at about pH 7-9.

19. The method of claim 1, wherein step (a) comprises diluting the sample with tris EDTA buffer at about pH 7-9.

20. canceled

21. The method of claim 1, wherein the amplification reaction is a polymerase chain reaction-based amplification reaction.

22. canceled

23. The method of claim 1, further comprising detecting the target sequence after amplification.

24. The method of claim 1, further comprising detecting the target sequence after amplification with a nucleic acid construct.

25. The method of claim 1, further comprising detecting the target sequence after amplification with a labeled nucleotide probe.

26. The method of claim 1, further comprising detecting the target sequence after amplification with a fluorescently labeled nucleotide probe.

27. The method of claim 1, further comprising analyzing the target sequence after amplification.

28. The method of claim 1, further comprising determining the sequence of at least one nucleotide of the target sequence after amplification.

29. (canceled)

30. The method of claim 1, wherein the sample is a blood sample selected from the group consisting of: a serum sample, a plasma sample, and a whole blood sample.

31-46. (canceled)


SEQUENCE LISTING (7 sequences)

SEQ ID NO: 1 - DNA, 52 bp, Homo sapiens
ggggcctacacggatggccacagccagtcgcccgctgcgccccagatgagga

SEQ ID NO: 2 - DNA, 52 bp, Homo sapiens
agcactacaggcagcaccagcgaagagggccgtgacagccgttgccatctgc

SEQ ID NO: 3 - DNA, 51 bp, Homo sapiens
aaggatatagggaaaaccttgaaagygatgtctgtggtggccgtctttggc

SEQ ID NO: 4 - DNA, 51 bp, Homo sapiens
tacgaggagctggtgctagccttgckttccgaggaggacggcctggccgaa

SEQ ID NO: 5 - DNA, 51 bp, Homo sapiens
ccgtgacagccgttgccatctgctgmcggagccggcacctggcgcaggcct

SEQ ID NO: 6 - DNA, 51 bp, Homo sapiens
ggtactgacccccaacctggtggccrccctgccccccagcacccatggggc

SEQ ID NO: 7 - DNA, 51 bp, Homo sapiens
gagtgtggcctgtgcagaagggaccraggctggtgagaccaggagggcctg